(19) United States
(12) Patent Application Publication
(10) Pub. No.: US 2020/0131458 A1
(43) Pub. Date: Apr. 30, 2020

(54) PANEL FOR PHOTOBIOREACTOR AND METHOD FOR MANUFACTURING SAME

(71) Applicant: Brochier Technologies, Villeurbanne (FR)

(72) Inventors: Cédric BROCHIER, Lyon (FR); Laure PERUCHON, Villeurbanne (FR)

(73) Assignee: Brochier Technologies, Villeurbanne (FR)

(21) Appl. No.: 16/463,385

(22) PCT Filed: Sep. 28, 2017
(86) PCT No.: PCT/FR2017/052656

§ 371 Date: May 23, 2019

(30) Foreign Application Priority Data
Nov. 25, 2016 (FR) 1661481

(51) Int. Cl. C12M 1/00; C12M 1/12; C12M 3/00

(52) U.S. Cl. CPC C12M 21/02; C12M 21/08; C12M 23/22; C12M 23/04

(57) ABSTRACT

The invention relates to a panel (100, 200, 300, 400) for a photobioreactor comprising at least: two plates assembled together, at least one of which is transparent and between which a lighting device is inserted; at least two openings allowing the passage of a fluid from a first face to a second face of the panel (100, 200, 300, 400) and characterized in that said luminous device is a fabric (101, 201, 202, 301, 401) including at least one optical fiber (2) capable of diffusing the light through said at least one transparent plate.

### FIELD OF THE INVENTION

The present invention relates the field of lighting devices made from optical fibers. More specifically the invention is used as a lighting device for the culture of photosynthetic microorganisms within a photobioreactor.

### PRIOR ART

In the industry of photosynthesis by microorganisms, two techniques are mainly known:
techniques in open reactors; and
techniques in closed reactors.

The open reactor technique was very quickly set aside in favor of the closed reactor technique. In fact, the open reactor technique has many disadvantages for being implementable on an industrial scale. The major disadvantage is that of the contamination of the photosynthetic microorganisms and in particular by sources coming from the outside environment such as gases present in the air.

On the other hand, closed photobioreactors have many advantages given as examples and without limitation:
keeping photosynthetic microorganisms sterile;
reducing energy inputs;
increasing productivity.

Over recent years two categories of closed reactors were developed by industry, specifically those operating with natural light coming from the outside environment, and those operating with artificial light.

The first category of closed reactors is provided with transparent plates allowing light to pass so as to supply light energy to the microorganisms' culture medium. This type of reactor does not however provide optimal productivity conditions because of the low light level provided within the culture medium. To counter these deficiencies, some companies had the idea of developing devices for accumulating light energy on the surface of the reactors so as to redistribute it within the enclosure in particular by routing means such as raw optical fibers. Document FR 2 968 094 describes this principle of light capture and redistribution within the reactor in more detail. Just the same, the light supplied still seems insufficient for bulk production. Additionally, conditions inside the reactor contribute to the rapid breakdown of the lighting device within the photobioreactor.

A second category of closed reactors was developed which consists of producing artificial light within the reactor. To do that, the panels within the reactor are provided with light-emitting diodes (LED) or other equivalent members. Even though the ratio of lit surface to culture volume and therefore the light energy efficiency was significantly increased, these devices have a significant disadvantage, the breakdown of the unprotected lighting devices (LED). In fact, it was observed that the microorganisms have a tendency to clump near the LED which leads to deterioration thereof. Consequently, culturing photosynthetic microorganisms within these devices is limited.

The Applicant thinks that there is a clear need to develop an effective device for diffusion of light for the culture of photosynthetic microorganisms in photobioreactors.

In order to remedy these problems, it therefore proposes a panel for photobioreactors with which to resolve the aforementioned problems.

### BRIEF DESCRIPTION OF THE INVENTION

The invention relates to a panel for photobioreactors comprising at least:
two plates assembled to each other, at least one of which is transparent, and between which a lighting device is inserted;
at least two openings allowing the passage of a fluid from a first surface towards a second surface of the panel.

In accordance with the invention, this panel is characterized in that the lighting device is a textile incorporating at least one optical fiber which is capable of diffusing light.

In other words, the Applicant proposes a panel for a photobioreactor which comprises a light textile provided with optical fibers. These optical fibers are capable of diffusing light and do so laterally relative to their own length. In fact, when the optical fiber(s) is/are supplied with light from a light source, the light is then conducted along the fiber. This same light can then be partially or completely diffused near certain regions of the optical fiber. Plates of a transparent kind partially or completely transmit the light and do so towards a predetermined region. Further, with the plates the lighting device can be protected from all external constraints (e.g. water, microorganisms) that could contribute to the breakdown thereof. Also, a fluid can pass through the panel because of the openings in the panel, so that the volumes defined between two adjacent panels can be connected. Thus, this panel can therefore be implemented as a lighting device in particular within culture enclosures for photosynthetic microorganisms like, for example, photobioreactors.

In practice, several embodiments for the panel are conceivable, in particular depending on:
the layout and/or the structure of the lighting device;
the layout of the protective plates;
the layout of the optical fibers within the fabric;
the mode of diffusion of the light;
the color of the light;

and therefore more generally the desired application.

As already explained, the lighting device is a textile incorporating at least one optical fiber which is capable of diffusing the light, in particular transversely.

In practice, this textile can just as well be made in the form of a fabric, knit or braid. Generally, the light textile is preferably a fabric which is composed of a warp and weft of yarns arranged according to predetermined patterns that the person skilled in the art will know how to determine according to the applications.

Advantageously, this fabric results from a jacquard method during which the mode of distribution of the yarns of the warp and/or weft, but also that of the optical fibers, is precisely controlled. Thus the optical fibers are woven continuously and identifiably within a textile core.

In practice, the fabric also has binding yarns with which to hold the optical fibers within the woven textile core. It involves yarns of the warp when the optical fiber is inserted in the weft or yarns of the weft when the optical fiber is in the warp. However, the optical fiber is preferably inserted in the weft and in this case, the binding yarns are warp yarns.

Further, the textile advantageously has binding yarns distributed on the optical fibers according to a satin type weave so as to optimize the surface for diffusion from the optical fibers.

The lighting device can have various layouts and have them according to the targeted applications.

According to a first preferred embodiment, the lighting device is a complex of two superimposed fabrics. These fabrics can be identical or different according to the applications.

Generally, the woven assembly thus comprises a juxtaposition interface formed between the surfaces of the two fabrics in contact. The assembly also has two surfaces which are opposite and which are each in direct contact with the protection plates, respectively a first plate and a second plate.

According to a first embodiment, the two fabrics are superimposed one on the other and held by addition of the two protective plates.

According to a second embodiment, the adhesion between the two fabrics is ensured by an adhesive thermoplastic film. It can advantageously be chosen among polyurethane (PU) or any other resin thermofusible when hot, in the temperature range recommended for optical fibers, which is under 80° C.

The adhesive film has optical properties towards light which depend on the targeted applications. According to a first variant, the adhesive film can be transparent to light. In this case, the film does not block the light and allows it to pass nearly completely. Thus, the light released by a fabric towards the transparent film is partially transmitted by the directly opposite fabric, to then be diffused towards the zone intended to be lit. Under these conditions, the panel reduces light energy losses by the layout and composition thereof.

According to a second variant, it may be partially opaque to light, but reflecting. In this case, the film preferably has a white color.

Advantageously and for greater stiffness, the two fabrics can be separated from each other by a stiff or semi-stiff spacing element made of transparent polymer, which is advantageously chosen from the group comprising polymethylmethacrylate (PMMA), polyurethane (PU), polycarbonate (PC), polyvinyl carbonate (PVC), polypropylene (PP) and cellulose acetate.

Further, the spacing element advantageously has a thickness included between 100 and 500 μm, and more advantageously between 150 and 300 μm.

In this case, adhesive materials are then preferable in order to be able to provide the adhesion of each of the fabrics on each side of the spacing element. Further, the adhesive material and the spacing element preferably have compatible adhesive properties in order to provide the preferred adhesion.

In practice, the optical fibers are advantageously located near the surfaces of the fabric which are directly in contact with the protective plates. In other words, the optical fibers emerge slightly from the surface of each of these surfaces.

Quite obviously, the person skilled in the art will know how to adapt the thickness of each of the fabrics so as to enhance one mode of light diffusion over another.

In other words, the person skilled in the art will know how to change the appropriate parameters for each mode of light diffusion in particular by working with parameters such as the nature and thickness of the materials and also the light diffusion properties thereof, etc.

In practice, the overall thickness of the panel is advantageously included between 1 and 5 mm, more advantageously included between 1.5 and 3 mm.

According to another embodiment, the lighting device consists of a single-layer fabric advantageously produced by a jacquard method.

This fabric advantageously has a weave chosen among satin, twill and taffeta, preferably a satin type weave.

In this case, the optical fibers are woven within the fiber core and consequently they emerge advantageously on either side of the plane formed by the fiber core. Further, the optical fibers tend to emerge principally and preferably from one side of the fabric instead of the other.

In practice, the thickness of this fabric is included between 0.5 and 5 mm, advantageously included between 0.5 and 3 mm.

As already explained, a spacing element can also be added to increase the stiffness of the textile inserted between the two protective plates. Of course, the protective plate is made of a material having adequate optical properties.

Depending on the applications, the person skilled in the art will know how to adapt the textile thickness so as to enhance one mode of light diffusion over another.

According to a third embodiment, the lighting device is a single-layer fabric folded on itself within which all the optical fibers are arranged on the opposite facing surfaces after folding. In other words, the fabric is folded on itself such that all the optical fibers which are on a single surface of the fabric, are located on the outer side from the fold.

As previously indicated, the fabric advantageously has a weave chosen among satin, twill, and taffeta, more advantageously satin.

Further, a spacing element can be inserted between the two portions of the folded fabric so as to increase the stiffness of the lighting device. As previously indicated, this spacing element advantageously has a thickness included between 100 and 500 μm, and preferably between 150 and 300 μm. Further, it advantageously shows properties of transparency to light; preferably it transmits at least 80% of the light.

There again, the person skilled in the art will know how to adapt thickness and fabric parameters and also the light diffusion properties of the materials used.

The optical fibers are capable of diffusing light laterally because they have previously undergone a surface treatment with which to make surface modifications.

“Surface modifications” is understood as meaning any modifications of the geometry and/or physical and chemical properties of the surface of the optical fibers, obtained by mechanical, thermal or chemical treatment, so that light propagating within the fiber can escape therefrom near said surface modifications.

According to one embodiment, the surface treatment may be mechanical treatment by abrasion. The texture of the surface of the optical fibers can be modified by mechanical treatment by abrasion. A part of the light is thus diffused from the surface modifications giving the optical fibers lateral lighting properties. This type of treatment is advantageous because the distribution of surface modifications along the lighting device can be controlled, by adapting the abrasion speed, rate and pressure. Thus, more particularly a number of surface modifications can be formed, which depends on the light intensity within the device and the preferred distribution.

As a variant, the surface treatment may also be performed during an optical treatment by laser radiation. A number of surface modifications depending on the intensity of the light available within the device and the length of the optical fibers can be obtained with treatment by laser radiation.

Advantageously in practice, the number of surface modifications on the sheath of the optical fibers, per unit of length, increases over the length of the fiber going from the first end facing the light source towards the opposite second end. With this distribution of surface modifications, a maximum of light can be conducted while providing homogeneous light diffusion along the full length of the optical fibers.

According to a specific embodiment, the optical fiber is connected to a light source at each of the ends thereof. In this case, the surface treatment can be adapted such that:
a first light source present at a first end is able to light a first half-length of the optical fiber; and
a second light source positioned facing the second end of the optical fiber is able to light the second half-length of the optical fiber.

In all cases, with such a surface treatment a culture of microorganisms which is mostly uniform over the entire volume considered can be ensured.

More generally, the surface treatment of the optical fibers can also be adapted depending on the quantity of light diffused in the privileged areas of the volume considered. In other words, the person skilled in the art will know how to adapt the surface treatment of the optical fibers depending on the applications.

Further, the optical fibers are advantageously made of a polymer material chosen among polymethylmethacrylate (PMMA) and polycarbonate (PC).

Within the textile and depending on the applications, the optical fibers can be grouped or independent of each other. When they are grouped, they can equally well be in one single bundle or in a plurality of sub-bundles.

The optical fibers from one bundle or from each sub-bundle can be gathered at least one end thereof in a ring intended to be combined with a light source.

In general, the optical fibers are supplied by at least one light source. The light source may be either natural (for example coming from the sun) or artificial depending on the applications. Further, this light source is advantageously outside of the panel.

In some cases, the light source can emit white light or a spectral color according to the applications. In other cases, the light source can emit UV, advantageously UV-A with wavelength included between 315 and 400 nm.

It can be a single light source shared with all the optical fibers. Or instead, it can be a plurality of light sources that are identical or different and specific to each of the optical fibers. In practice, the one or more light sources can be chosen among light-emitting diodes, organic (OLED) and polymeric (PLED or P-OLED) light-emitting diodes, laser diodes or combinations thereof.

Depending on the applications, the panel diffuses the light along preferred directions and ways. Regardless of the layout of the lighting device previously described, the panel from the invention is able to diffuse the light according to at least one of the following embodiments.

According to a first embodiment, the two-sided lighting panel has two fabrics assembled back to back. Since each fabric essentially comprises all the optical fibers on a single surface, the one-piece fabric emits light from one side of the fabric. In practice, the one-piece fabric emits between 70 and 90% of the total light from one side of the fabric and diffuses 10 to 30% of the light from the other side of the fabric. Since the lighting device comprises two back-to-back fabrics, it diffuses light from both sides of the panel substantially equivalently. In practice, the lighting device diffuses on each of the two sides between 80% to 120% of the light of a one-piece fabric, from the combination of the light from the two fabrics.

According to a variant in which the fabric comprises an equal share of optical fibers on each of the surfaces thereof, the lighting device diffuses the light from both sides of the panel. In other words, 50% of the total light is emitted from each side of the panel.

According to another embodiment, the lighting device emits light solely from one side of the panel. In all cases, a very small quantity of light is diffused from the other side of the panel. This type of panel is in particular attractive for use as an outside panel in a photobioreactor. An outside panel indicates a panel which contributes to the bearing structure of a structure such as a photobioreactor. In fact, only one side of the panel is intended to diffuse light towards the volume of the culture; the other side plays the role of bearing structure for the photobioreactor.

Independently of the configuration of the lighting device, the panel can be monochrome or polychrome according to the applications. “Monochrome panel” means a panel which diffuses light of a single color, for example, white, blue or red. Conversely, “polychrome panel” designates a panel which diffuses light of several different colors. In other words, the panel can for example diffuse blue and red light simultaneously. Of course, the nature of the emitted light will depend on the choice of the light source as already explained above.

According to a first embodiment, when the optical fibers are independent of each other, they can either all be supplied by an identical light source, or each be supplied by one light source emitting over various ranges of wavelengths.

According to a second embodiment, when the optical fibers are grouped at least one end thereof into one or more bundles which each emerge into a ring, several lighting variants are then possible.

In practice, if the fibers are gathered into just one bundle, just one light source, of any color, is then needed.

Instead, when the optical fibers are gathered in a plurality of bundles, each bundle can be lit by an individual light source; together these light sources can be identical or different colors according to the applications.

In order to be able to implement a photobioreactor for example, the lighting device must be protected in particular from water or even from microorganisms. The protection can be provided by various means described below.

In every case, the lighting device can be arranged between two protective plates which are transparent and, importantly, inert to water.

In the remainder “transparent” plate means a plate able to transmit advantageously between 80 and 100% of the light which it receives, and more advantageously between 90 and 100%.

In practice, the plates intended to sandwich the lighting device are transparent. Of course and according to the targeted application, they have light transmission properties which can be identical or different.

Further, these two plates can be made of glass or of a polymer lighter than glass. The polymer has excellent light transmission properties and is advantageously chosen among polymethylmethacrylate (PMMA) and polycarbonate (PC), preferably polymethylmethacrylate (PMMA).

Further, these plates are advantageously inert to the surrounding material. In fact, they serve to protect the lighting device, in this case the textile, during use thereof. In other words, the purpose of these plates is to protect the textile lighting device from all the elements present in the photobioreactor.

According to a preferred embodiment, the plates are made of a single material chosen among the materials indicated above. They preferably transmit between 80 and 100% of the incident light received, more preferably between 90 and 100% and in the example of PMMA, about 92%.

Further, these plates are in practice always secured to each other and the securing is done by adhering, screwing or any other methods that are appropriate and known to the person skilled in the art.

In general, a photobioreactor comprises a plurality of lighting panels mounted in stacks within an appropriate enclosure. The arrangement of lighting panels serves to define microorganism culturing zones between the panels.

According to a first embodiment, a culture zone is formed by the space defined between two successive light panels separated by a peripheral framework, for example of PVC, defining a volume in which fluid flows. In this case, the panel is structurally composed of a lighting device sandwiched between two protective plates which can have various configurations depending on the applications.

The protection of the lighting device can be provided by machining at least one of the two protective plates so as to form a housing capable of receiving the lighting device. In a first variant, both protective plates are identically machined to form a housing with a volume substantially equal to half the volume necessary to confine the lighting device. In this case, the advantage resides in the implementation of a method with which to obtain identical plates. In another variant, a machined protective plate is placed facing an intact protective plate. In this case, the lighting device comes to be housed in the space formed within the machined protective plate. In practice, the dimensions of the lighting device are advantageously identical to that of the space.

The protection can also be implemented by adding at least one spacer of thickness similar to that of the lighting device and inserted between the two protective plates, so as to surround the lighting devices for isolating them from the medium outside the panel.

These spacers can be independent of the plates and held in position while the two plates are brought into contact with each other, or dependent on the plates by adhering. They can also be arranged either on a single protective plate or on both protective plates.

Further and according to the applications, these spacers can be made of several independent segments arranged butting together around the lighting device, or can take on the shape of a single frame surrounding the lighting device.

These spacers may comprise a groove in order to house a sufficiently compressible joint for providing intimate and continuous contact with the plates during tightening of the plates, in the interest of a tight seal. Preferably, the spacers are made of a material similar to that of the plates in order to ensure compatibility between the adhesive, plate and spacer during assembly of the spacers with the plates.

Generally, each spacer or portion of spacer placed facing an opening of one of the protective plates also has an opening which is identical thus continuous allowing the passage of the fluid from a first surface towards a second surface of the panel.

In the goal of manufacturing identical plates, the means forming the spacers are advantageously distributed on both plates of a single panel, and arranged on the facing surfaces of these two plates. Each plate preferably has means forming identical thickness spacers. Such that, when the two plates are assembled, they are separated by a distance corresponding to the sum of the thicknesses of the spacers of both plates. This distance then corresponds to the thickness of the lighting device.

According to a particular embodiment, the immobilization of the lighting device between the two protective plates can be implemented by combining the machining of the protective plates and the use of spacers.

According to a second embodiment, the photobioreactor is composed of lighting panels adhered to each other which are configured to define a volume for passage of the fluid inside the overall volume thereof. This volume can be defined, either in one of the protective plates, or in a structure forming a chassis which supports the lighting devices protected by the protective plates.

The chassis is preferably transparent and has a hollow space with a thickness defining the dimension of the culture volume. In this case, since the culture zone is directly incorporated in the panel, it is sufficient to bring the panels in contact with each other in order to obtain the photobioreactor. Further, this overcomes the placement of a peripheral frame between panels which makes it possible to achieve a simpler and more compact stacking.

Generally, the chassis has a compartment in which the previously described plate/textile/plate assembly comes to be housed. In general, the assembly is arranged within the chassis by adhering using an adhesive transparent to light. The compartment can be formed in various ways depending on the applications: either by adding spacers or by machining in the thickness of the chassis. In the case of added spacers, they can be made either of a material identical to the chassis so as to form a monolithic structure, or of a different material leading to a heterogeneous structure.

Also, the movement of a fluid from a first surface towards the second surface of the panel can be ensured in various ways in particular by adjustment of the shape of the chassis.

According to a first variant, the chassis has two openings in the structure thereof: one near the upper part thereof and another near the lower part thereof. Advantageously these openings are formed in each of the projections which form the compartment intended to receive the assembly. This panel further comprises means for sealing in particular near the interfaces between the chassis and the assembly which could potentially be exposed to water and/or algae but also near the spacers, when they are used, intended to be in contact with the adjacent panel.

According to a second variant, the openings are defined jointly by the protective plate and the projections from the chassis arranged facing.

Because of the substantially central positioning of the assembly relative to the projections, two openings can be formed respectively situated between:
the upper edge of the assembly and the upper part of the chassis;
the lower edge of the assembly and the lower part of the chassis.

In this case, the portions of the plate/textiles/plate assembly, which define the openings with the chassis, are coated by means for sealing so as to isolate the lighting device from the fluid.

The panel can comprise an attachment member which serves to keep it on the bearing structure of the photobioreactor. This attachment member can be secured to the panel, but also removable. With this attachment member, the panel can advantageously be mounted on rails for guiding the bearing structure so as to maintain a relative positioning between the panels within the photobioreactor.

In general, the lighting device is not in practice fully confined between the two protective plates. Thus, the optical fibers and/or the optical fiber bundles emerge near the lateral parts of the plate/textiles/plate assembly which are perpendicular to the direction of the fabric weft. They generally do not have sealing means because they are not exposed to either the water or the microorganisms.

According to a specific embodiment, the lighting device is coated at least on one surface by a coating layer. Advantageously this coating layer is made from a polymer chosen among polyurethane, silicone, epoxy resin or any other hydrophobic material having adequate optical properties.

Further, depending on the targeted application, a panel is conceivable in which the fabric is protected on one side by a protective plate as described above and protected on the other side by a protective layer that is preferably transparent and inert against water.

A second aspect of the invention relates to the method for production of the panel previously described.

The method comprises the following steps:
a) a fabric is formed comprising:
   warp and weft yarns that form the core of the fabric;
   optical fibers woven in weft and/or warp within the fabric; and
   binding yarns forming part of the warp and/or weft yarns, maintaining the optical fibers inside the fabric;
b) the optical fibers receive a surface treatment so as to create surface modifications making the fabric suitable for emitting light;
c) at least one previously formed fabric is inserted between at least two protective plates;
d) the protective plates are attached to each other and a light panel is formed.

Optionally, the method can also comprise steps of gathering at least one of the floating ends of the optical fibers into at least one bundle in order for insertion into a ring. As a variant, the optical fibers can be treated before being woven.

Attachment step d) can be done either by using adhesive or by mechanical means, such as screws, for example. In this case, the seal of the panel is provided by the presence of previously described means of confinement.

As already explained, the lighting fabric can have several configurations. Consequently, the person skilled in the art will know how to adapt the method to each of the previously described configurations.

As an example, when the lighting device is composed of two fabrics joined back to back, the person skilled in the art will know to incorporate steps of positioning the fabric between the two plates with or without the addition of a spacing element and/or an adhesive material such as previously described. The same applies for the configuration involving a fabric folded on itself.

In the preceding it can be seen that the panel from the invention has many advantages and it can provide in particular:
use in a photobioreactor;
homogeneous and satisfactory lighting over the entire panel;
simple adaptation depending on the applications.

### BRIEF DESCRIPTION OF THE FIGURES

The invention will be better understood upon reading the following description, given solely as an example, and made in connection with the accompanying figures, wherein:

FIG. 1 is an exploded schematic view of a panel incorporating a single fabric according to a specific embodiment of the invention;

FIG. 2 is an exploded schematic view of a panel provided with a double fabric superimposed back-to-back according to another embodiment of the invention;

FIG. 3 is an exploded schematic view of the panel provided with the fabric folded on itself according to FIG. 1;

FIG. 4 is an exploded schematic view of the panel arranged within a chassis according to a specific embodiment of the invention;

FIG. 5 is a schematic section of a monochrome panel provided with a fabric according to any one of FIGS. 1, 2, 3 and 4;

FIG. 6 is a schematic section of a polychrome panel provided with a fabric according to any one of FIG. 1, 2 or 3 and lighting according to a first mode of operation;

FIG. 7 is a schematic section of a polychrome panel provided with a fabric according to any one of FIG. 1, 2 or 3 and lighting according to another mode of operation;

FIG. 8 is a schematic section of a double connection panel.

### METHOD FOR IMPLEMENTING THE INVENTION

The invention therefore relates to a lighting panel for a photobioreactor where the configurations of the lighting device lead to many embodiments.

As shown in FIG. 1, the panel 100 comprises at least:
two plates 41, 42 of which at least one is transparent:
   the first plate 41 comprises two openings 411, 412 and two spacers 50, 51 each having one opening, respectively 501 and 511;
   the second plate 42 comprises two openings 421, 422 and also two spacers 52, 53 each having one opening, respectively 521 and 531;

a fabric 101 inserted between the plates 41, 42; the lighting device 5 is a textile 100 incorporating at least one optical fiber 2 able to diffuse light through at least one transparent plate.

This fabric 101 has a satin weave made from warp yarns 12 and weft yarn 11 of Trevira® CS polyester. These yarns 11, 12 advantageously have flame-retardant properties. The yarns 11, 12 can however also be made from polyamide, fiberglass, or other synthetic fiber yarns, even metallic yarns. In general, yarns 11, 12 can be made from yarns having a yarn size included between 20 and 500 decitex.

In this case, the optical fibers 2 woven in weft are held within the fabric 101 by binding yarns 3. The binding yarns are positioned on the optical fibers according to the selected weave. The yarns 3 correspond to the warp yarns 12 which, in addition to contributing to the formation of the weave, serve to hold these optical fibers 2 within the fabric. The fabric 101 has between 7 and 15 optical fibers 2 per centimeter depending on the diameter of the optical fiber.

The fabric 101 has a thickness which is included between 0.5 and 3 mm, and which is a function of the diameter of the optical fiber and the mode of diffusion of the light.

In fact and according to the embodiments, the fabric can be suited for emitting:
from a single side of the fabric 100; or
from both sides of the fabric 100.

The person skilled in the art will therefore know how to adapt the thickness of the fabric and also the weaving methods to give this fabric the desired properties for diffusion of the light depending on the applications.

The lighting fabric 101 can be protected with a system of plates 41, 42. In fact, the fabric 101 is sandwiched between two chemically identical plates 41, 42 preferably made of polymethylmethacrylate (PMMA) or glass.

In practice the plates 41, 42 are intended to be assembled to each other and sandwich the light fabric 101 to form the panel 100. The plate 41 is provided with a spacer 50 mounted horizontally on the upper part thereof. The orifice 501 of the spacer is facing the opening 411 of the plate 41. The plate 41 also has a spacer 51 mounted horizontally on the lower part thereof. There again, the orifice 511 of the spacer 51 is positioned facing the opening 412 of the plate 41.

Conversely, the plate 42 is provided with a spacer 52 mounted horizontally on the lower part thereof. The orifice 521 of that spacer 52 faces the opening 421 of the plate 42. The plate 42 also has a spacer 53 also mounted horizontally on the lower part thereof. The orifice 531 of this spacer 53 faces the opening 422 of the plate 42.

A transparent adhesive compatible with the optical fibers secures the plates 41, 42. The fabric 101 is partially confined and blocked between the two plates 41, 42 which thus provide protection therefor.

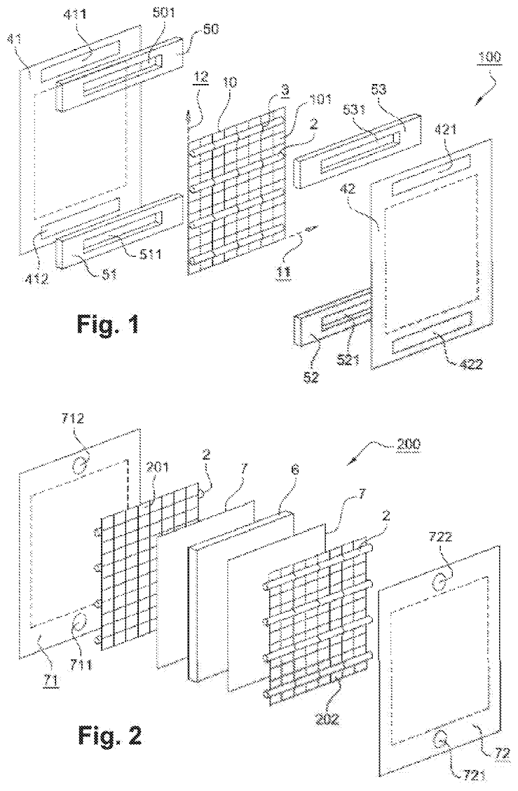

Unlike FIG. 1, the panel 200 shown in FIG. 2 relates to the embodiment of a lighting device comprising two fabrics 201, 202 superimposed back to back. In this case, the fabrics 201, 202 are identical.

Further, a transparent, stiff or semi-stiff spacing element 6 is positioned on the superimposition interface of the two fabrics 201, 202. The element 6 is advantageously made from a sheet of transparent PMMA polymer and gives the lighting device an increased stiffness.

A double-sided adhesive material 7 made of polyurethane can also adhere the fabrics 201, 202 at each of the surfaces of the spacing element 6.

The lighting device is sandwiched between a first plate 71 having two openings 711, 712 and a second plate 72 having two openings 721, 722. When the plates 71 and 72 are brought into contact, the openings 711 and 721 and the openings 712 and 722 are arranged directly opposite and continuously.

Here again according to the embodiments, the fabric can be suited for emitting:
from a single side of the panel 200; or
from both sides of the panel 200 in identical quantity.

However, the panel 200 has a specific interest when it is used as a lighting device for two-sided diffusion of the light. In other words, the panel 200 can be used within a photobioreactor for bringing light simultaneously to two sites for culturing microorganisms located respectively on each side of the panel 200.

Further, the optical properties of the spacing element 6 and the adhesive material 7 are decisive in the mode of diffusion of the final panel 200.

Again, the person skilled in the art will be able to determine the properties for the panel depending on the preferred mode of diffusion of the light.

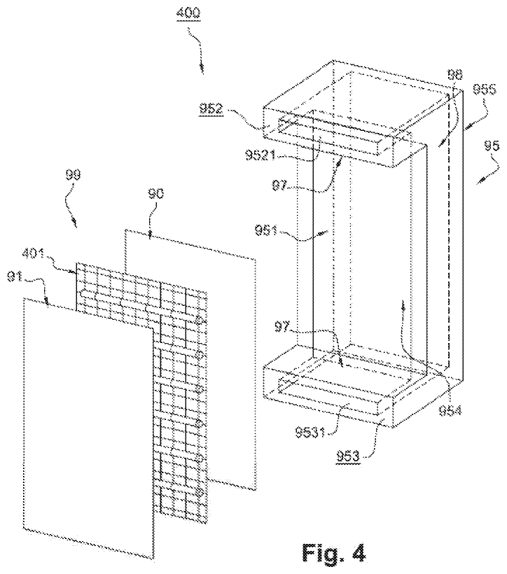

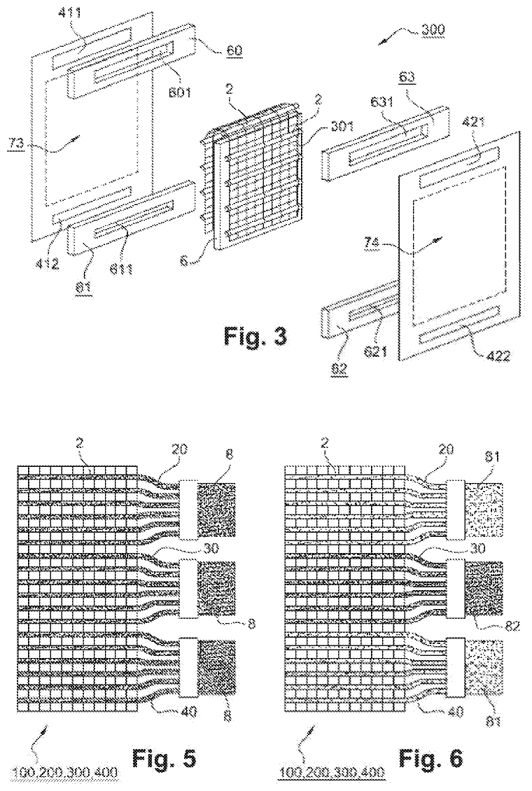

As shown in FIG. 3, the panel 300 comprises a fabric 301 folded back on itself.

As already explained, this fabric 301 can incorporate in the area of the folding zone thereof a spacing element 6 such as previously described.

This embodiment of the panel 300 is able to operate according to the mode of two-sided diffusion of the light.

Further, the projections 60, 61, 62, 63 surround the thinnest central region, made by machining the thickness of the plates 31, 32. They contribute at least partially to the confinement of the fabric 301 and especially serve to isolate it from the outside environment. The projections 60 and 63 in the upper part of the plates 73 and 74 each have an opening 601 and 631 that is wider than the openings 611 and 621 of the projections 61 and 62 located respectively in the bottom part of the plates 73 and 74. Of course, the opposite configuration, not shown on the figures, is also conceivable.

In FIG. 4, the panel 400 comprises a single fabric 401 inserted between two protective plates 90, 91 of PMMA to form an assembly 99. The fabric 401 is adhered to each of the two plates 90, 91 and over its full periphery. This assembly 99 is adhered within a compartment 951 of the chassis 95 also made of PMMA. With adhesive, the seal between the assembly 99 and the chassis 95 in the area of the interfaces 97 is ensured. The chassis 95 has projections 952, 953 which define the height of the compartment 951 intended to receive the assembly 99. This height is identical to that of the assembly 99. Further, the width the compartment 951 is identical to that of the assembly 99. Thus the assembly 99 is perfectly housed within the compartment 951 with appropriate means present for sealing. Since the projections 952, 953 are also made of PMMA, the chassis 95 therefore has a monolithic structure. Further, each of the projections 952, 953 has an opening, respectively the opening 9521 and the opening 9531, allowing the passage of a fluid from one surface of the panel to the other surface.

Further, the chassis 95 is hollow and has a wall 954 on which the assembly 99 is placed and a hollow volume 98 located behind the wall 954.

When this panel 400 is used in a photobioreactor, the water flows into the projections 952, 953 along the openings 9521, 9531 and then continues its flow in the hollow space 98 of the chassis 95. This hollow space 98 corresponds in practice to the culturing zone for the microorganisms intended to be cultivated.

A panel was described in which the chassis has a monolithic structure, however the person skilled in the art is able to make a chassis with heterogeneous structure. For example, a variant not shown consists of a chassis comprising a compartment intended to receive the assembly and whose projections are formed by the addition of spacers made from a different material than that of the chassis.

Another variant not shown consists of a panel for which the openings are defined jointly by the protective plates and the projections from the chassis arranged facing.

FIG. 5 shows a panel 100, 200, 300, 400 according to the invention lit by a uniform red light source.

As already explained, the person skilled in the art will know how to determine the structural layout of the fabric depending on the targeted applications. Further, the person skilled in the art will also be able to choose the nature of the light source according to the applications.

In the following, the invention is illustrated without limitation by three variants that differ from each other by the mode of connection to the light sources.

As shown in FIG. 5, the optical fibers 2 are grouped in three distinct bundles 20, 30, 40. Each of these bundles 20, 30, 40 comprises between 18 and 154 optical fibers depending on the diameter of the fiber and the source. The panel 100, 200, 300, 400 diffuses red light. Of course, by a simple change in the nature of the light source 8, the panel 100, 200, 300, 400 could then diffuse a different color light.

As shown in FIG. 6, the optical fibers 2 are again grouped in three bundles 20, 30, 40. The difference lies in the alternation of the colors diffused by the panel 100, 200, 300, 400. In fact, bundles 20 and 40 diffuse white light whereas bundle 30 defuses a red light. Of course, the person skilled in the art will be capable of varying the colors emitted by the panel 100, 200, 300, 400.

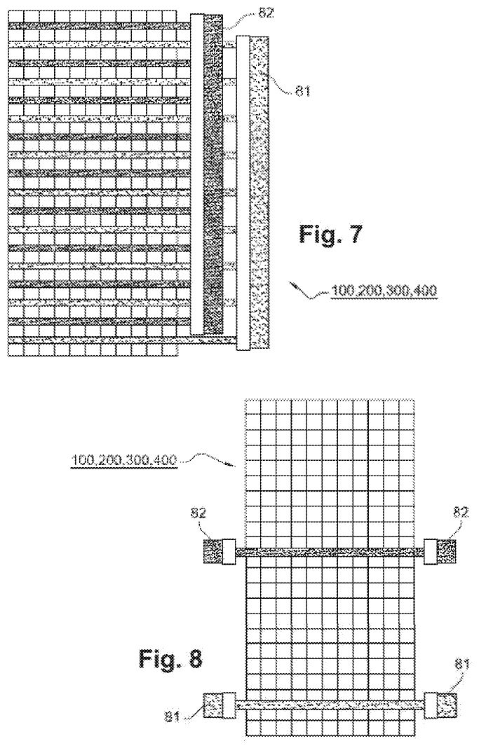

As shown in FIG. 7, the optical fibers are connected in two distinct bundles, advantageously by grouping every other fiber into each bundle. The bundles are supplied respectively by a blue light source 81 and a red light source 82. Preferably, each bundle groups between 18 and 154 optical fibers.

Generally, each optical fiber 2 can be supplied at either one or both ends. When the optical fiber is lit at each of these ends, 100% of the light emitted by the light source is used.

As shown in FIG. 8, the panel 100, 200, 300, 400 has optical fibers 2 which are connected at each end thereof respectively to a light source 81, 82.

In general, the light source 8 is advantageously an LED which can emit over the full visible spectrum. Preferably, when an optical fiber 2 or bundle of optical fibers 2 is supplied by a single light source 8, this source 8 has a power included between 3 and 10 W, advantageously around 10 W. Inversely, when an optical fiber 2 or bundle of optical fibers 2 is lit at each of the ends by a light source 8, and this case the light source 8 has a power included between 1 and 3 W.

Generally, the distance separating the various light sources 8 would supply the optical fibers 2 or the bundles of optical fibers 2 is included between 1 and 10 cm, where the distance is determined depending on the diameter of the fiber and the source.

1. A panel (100, 200, 300, 400) for photobioreactor comprising at least:
two plates assembled to each other, at least one of which is transparent, and between which a lighting device (5) is inserted;
at least two openings allowing the passage of a fluid from a first surface towards a second surface of the panel (100, 200, 300, 400) and characterized in that the lighting device is a textile (101, 201, 202, 301, 401) incorporating at least one optical fiber (2) able to diffuse light through at least one transparent plate.

2. The panel according to claim 1, characterized in that said textile is a single-layer fabric (101).

3. The panel according to claim 1, characterized in that the textile is a single-layer fabric (301) folded on itself within which all the optical fibers (2) are arranged on the opposite facing surfaces after folding.

4. The panel according to claim 1, characterized in that said textile is a complex of two identical, superimposed fabrics (201, 202).

5. The panel according to claim 4, characterized in that said two fabrics are separated from each other by a stiff spacing element (6) made of transparent polymer, which is advantageously chosen from the group comprising polymethylmethacrylate (PMMA), polyurethane (PU), polycarbonate (PC), polyvinyl carbonate (PVC), polypropylene (PP) and cellulose acetate.

6. The panel according to claim 1, characterized in that said textile (101, 201, 202, 301, 401) diffuses light from each side of said panel.

7. The panel according to claim 6, characterized in that said textile (101, 201, 202, 301, 401) diffuses between 70 and 90% of the total light from one side of said panel, and between 10 and 30% from the other side of said panel.

8. The panel according to claim 7, characterized in that said textile (101, 201, 202, 301, 401) diffuses 50% of the total light from each side of said panel.

9. The panel according to claim 1 characterized in that said optical fibers (2) are grouped at one end thereof into at least one bundle of optical fibers (2) which emerge in one ring, where said bundle is lit by at least one light source (8).

10. The panel according to claim 1 characterized in that said plates are made of a material chosen among polymethylmethacrylate, polycarbonate, and glass, advantageously polymethylmethacrylate.

11. The panel according to claim 1 characterized in that the plates are identical and have two openings each.

12. The panel according to claim 1 characterized in that it comprises a chassis (95) on which the two plates are secured and between which the lighting device is inserted.

13. The panel according to claim 12, characterized in that said chassis has two openings (9521, 9531).

14. The panel according to claim 12, characterized in that two openings are defined between respectively:
the upper edge of the plates and the upper part of the chassis;
the lower edge of the plates and the lower part of the chassis.
